(19) United States
(12) Patent Application Publication
(10) Pub. No.: US 2007/0276291 A1
(43) Pub. Date: Nov. 29, 2007

(54) Palpation device

(75) Inventors: James R. Hewit, Dundee (GB); Alan P. Slade, Dundee (GB); Mark S. Pridham, Dundee (GB); Robert P. Keatch, Dundee (GB)

Correspondence Address: GIFFORD, KRASS, SPRINKLE,ANDERSON & CITKOWSKI, P.C, PO BOX 7021, TROY, MI, 48007-7021, US

(73) Assignee: UNIVERSITY OF DUNDEE, Dundee (GB)

(21) Appl. No.: 10/567,495

(22) PCT Filed: Aug. 6, 2004
(86) PCT No.: PCT/GB04/03384

§ 371 Date: Jul. 31, 2006

(30) Foreign Application Priority Data
Aug. 7, 2003 (GB) 0318498.3

(51) Int. Cl. A61B 5/103

(52) U.S. Cl. 600/587

(57) ABSTRACT

The invention relates to a palpation device and a palpation simulation device for use in surgery, such as minimal access surgery. The invention also relates to a method of palpating a body part, and to a method of simulating palpation of a body part. In one embodiment of the invention, a palpation device (10) is disclosed which comprises a first palpation assembly including a palpation member (12) and a light source in the form of an LED (14) and a second palpation assembly including a palpation member (16) and a light detecting means, in the form of a camera (18), for detecting light emitted by the light source (14). At least one of the first and second palpation members (12, 16) is movable with respect to the other member, to palpate a body part such as a patient's colon (20), disposed therebetween.

The present invention relates to a palpation device and a palpation simulation device, in particular, but not exclusively, to such devices for use in surgery, such as minimal access surgery. The present invention also relates to a method of palpating a body part, and to a method of simulating palpation of a body part.

Surgeons have the need to identify the condition of body organs and tissue to determine if they are healthy or not. This is normally done by the use of their tactile senses, that is they “palpate” the organ or tissue, often by squeezing and rubbing. They then analyse the results subjectively using their knowledge and skill. This process of intelligent palpation cannot be undertaken, in particular, during minimal access surgery (MAS). In MAS, the surgeon performs surgical procedures using long, slender instruments, which are passed into the body through small access wounds. Whilst this has undoubted benefits to the patient, in that the invasiveness of such procedures is much reduced, the surgeons are unable to get their fingers inside the body cavity due to the restricted access available. Thus there has been a considerable effort to try to return this useful sense of touch to minimal access surgeons by artificial means.

Most researchers have approached the field of artificial tactile sensing by trying to find a direct emulation of the way the surgeon works and have attempted to develop large arrays of elements, each capable of transducing the contact forces and pressure distributions set up between the touched surface and the sensing elements. This has proved to be very difficult due to the need to design very small, very sensitive and very robust elements. The elements comprising the array must also be safe to insert into the body cavity.

It is amongst the objects of embodiments of the present invention to obviate or mitigate at least one of the foregoing disadvantages.

Accordingly, the present invention provides a palpation device comprising:

a first palpation assembly including a palpation member and a light source; and

a second palpation assembly including a palpation member and light detecting means for detecting light emitted by the light source;

wherein at least one of the first and second palpation members is movable with respect to the other member, to palpate a body part disposed therebetween.

Advantageously, this allows a body part such as an organ, muscle, flesh, fat or other tissue to be palpated, whilst shining light through the body part and viewing its behaviour during manipulation. This may be particularly useful in identifying and classifying abnormalities such as tumours. In particular, this palpation of the body part, and thus any abnormality, may assist in initial location of the abnormality, and/or the measuring of one or more physical parameters of the body part/abnormality.

According to a second aspect of the present invention, there is provided a palpation simulation device comprising:

a first palpation assembly including a palpation member and a light source; and

a second palpation assembly including a palpation member and light detecting means for detecting light emitted by the light source;

wherein at least one of the first and second palpation members is-moveable with respect to the other member, to palpate a body part disposed therebetween.

References herein to palpate, palpating and palpation, are to the physical manipulation of a body part to determine physical/tactile parameters or properties of the body part, and to the simulation of such an exercise by manipulation and generation of an image of the behaviour of the body part during such manipulation.

Preferably, the device further comprises means for measuring one or more physical parameters or properties of the body part, such as the resistance to deformation in response to an applied force. The measuring means may comprise force measuring sensors for measuring the force applied to the body part to produce a deformation. The measuring means may further comprise software for performing feature recognition and classification based upon image analysis algorithms.

Preferably also, the first and second palpation members are independently moveable. This may advantageously allow the body part to be readily squeezed and/or rubbed between the first and second members. One or both of the first and second members may be moveable in at least two, and preferably three mutually perpendicular planes of motion, with respect to the body part. One or both of the first and second palpation members may be generally planar, and in particular may be in the form of a plate.

Preferably, the palpation device is for use in surgery, in particular minimal access surgery (MAS) where there is a restriction on the size of the entry wound which may be made.

The first palpation member may comprise a light transmitting member, particularly a transparent or translucent member. The first member may be of a glass or plastics material. The light source may be embedded in or physically/optically coupled to or adjacent the first member. Conveniently, the first member is optically shaped to transmit a substantial part of the light emitted by the light source through a surface of the first member adapted to be located adjacent the body part.

The second palpation member may comprise a light transmitting member, particularly a transparent or translucent member. The second member may be of a glass or plastics material. Alternatively, the second member may comprise a light sensitive charge coupled device (CCD). The CCD may form the whole of the second palpation member, or may be embedded in or physically/optically coupled or adjacent to the second member. The CCD may transduce the received light into a 2-dimensional (2-D) pixel array for output to a suitable display device, such as a monitor.

The light source may comprise a light emitting diode, optical fibres or any other suitable light source. The light detecting means may comprise a camera, endoscope, or a CCD, and may be embedded in or physically/optically coupled or adjacent to the second palpation member.

One or both of the first and second palpation members may be moveable between an insertion position and a use position. In the insertion position, the palpation device may be of reduced dimensions which may be advantageous for use in MAS. One or both of the first and second palpation members may comprise two or more planar members such as plates, pivotally coupled together for movement between the insertion and use positions. Thus a number of separate images of the body part may be obtained and patched together using, for example, suitable software.

The palpation device may be adapted to be mounted on support means, in use. This may be particularly advantageous in MAS, where space is restricted. The support means may comprise an arm including means for detecting the location and orientation of the palpation device. Preferably, the support means comprises a powered robot arm, but may alternatively comprise a manually operated arm.

The light source may be adapted to emit light of a frequency in the visible spectrum. However, the light source may be adapted to additionally/alternatively emit light of any suitable frequency, for example, structured light or light of a frequency in the infra-red spectrum.

The device may further comprise detecting means for detecting motion of at least part of the body part relative to one or both of the first and second palpation assemblies. In particular, the detecting means may be for detecting motion of at least part of the body part relative to the palpation members of one or both of the first and second assemblies. The part of the body part may comprise a tumour or other abnormality or the like which it is desired to detect. The detecting means may comprise at least one point of reference, and may in particular comprise a visible grid or the like provided on or in the/each palpation member. This may advantageously allow detection of motion of said tumour or the like relative to the/each palpation member, which may assist in determining more precisely the location of said tumour.

According to a third aspect of the present invention, there is provided a method of palpating a body part, the method comprising the steps of:

providing a first palpation assembly including a palpation member and a light source;

providing a second palpation assembly including a palpation member and light detecting means for detecting light emitted by the light source;

locating the first and second palpation assemblies with a body part disposed therebetween; and

moving at least one of the first and second palpation members relative to said other member, to palpate the body part.

According to a fourth aspect of the present invention, there is provided a method of simulating palpation of a body part, the method comprising the steps of:

providing a first palpation assembly including a palpation member and a light source;

providing a second palpation assembly including a palpation member and light detecting means for detecting light emitted by the light source;

locating the first and second palpation assemblies with a body part disposed therebetween; and

moving at least one of the first and second palpation members relative to said other member, to palpate the body part.

Embodiments of the present invention will now be described, by way of example only, with reference to the accompanying drawings, in which:

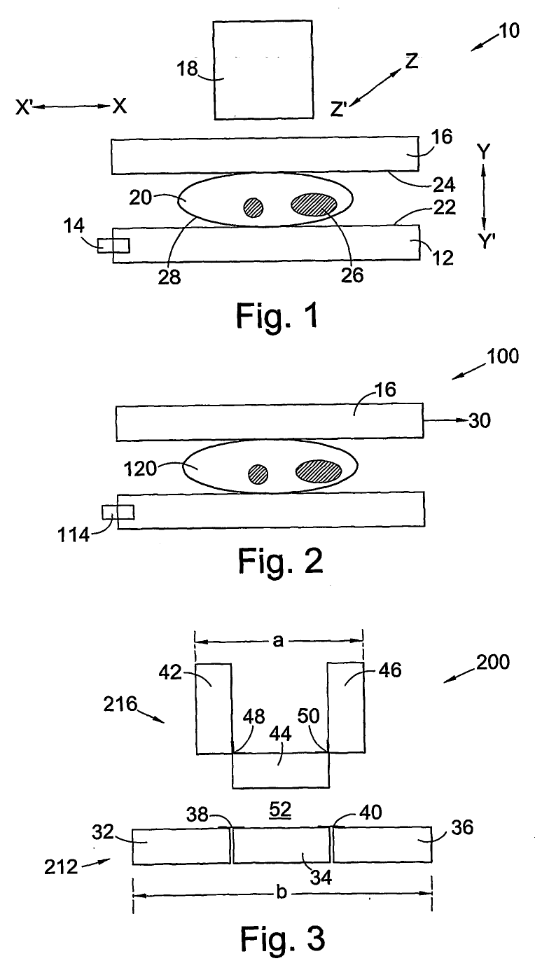

FIG. 1 is a schematic illustration of a palpation device in accordance with an embodiment of the present invention, shown in use;

FIG. 2 is a schematic illustration of a palpation device in accordance with an alternative embodiment of the present invention, shown in use;

FIG. 3 is a schematic illustration of part of a palpation device in accordance with a further alternative embodiment of the present invention;

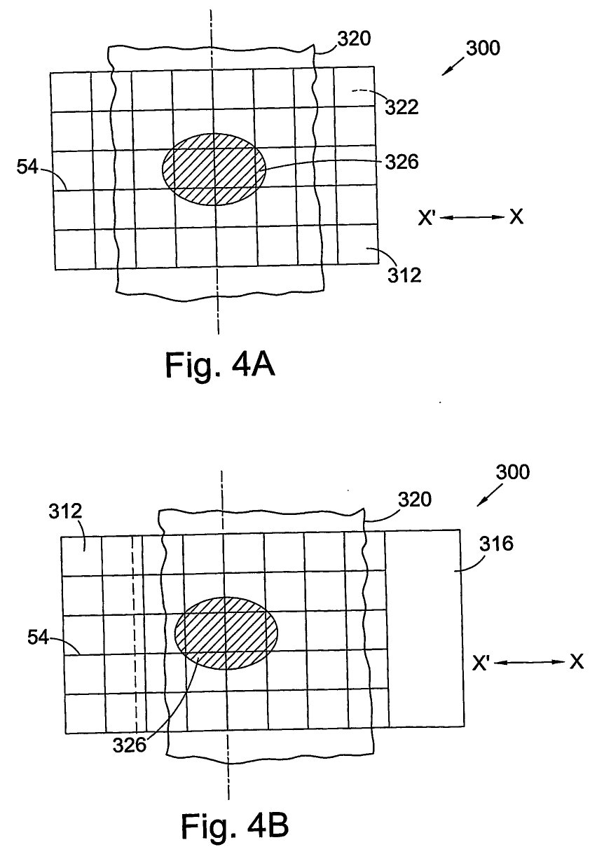

FIGS. 4A and 4B are schematic illustrations of part of a palpation device in accordance with a still further embodiment of the present invention, shown during palpation of a body part; and

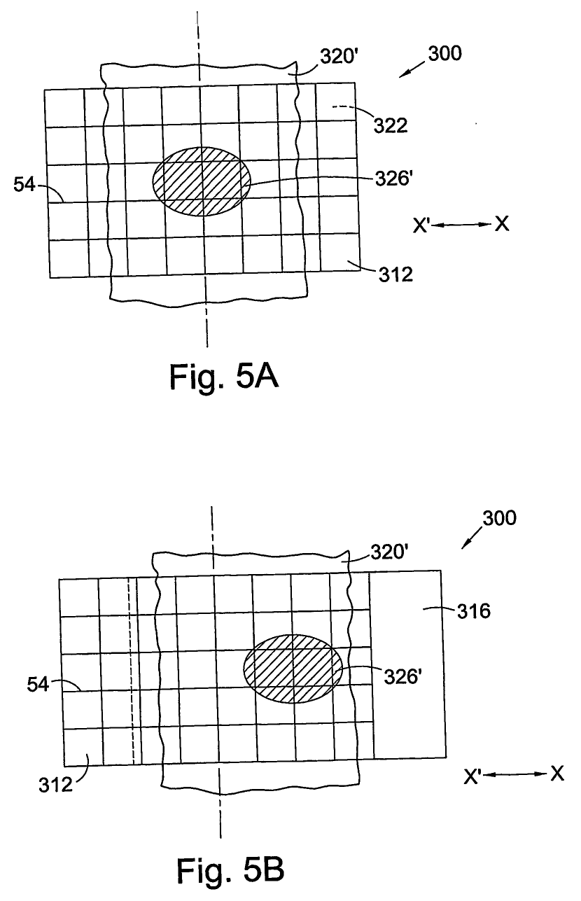

FIGS. 5A and 5B are views of the part of the palpation device of FIGS. 4A and 4B, shown at corresponding stages of palpation of a different body part.

Referring firstly to FIG. 1, there is shown a schematic illustration of a palpation device/palpation simulation device in accordance with an embodiment of the present invention, indicated generally by reference numeral 10. The palpation device 10 generally comprises a first palpation assembly including a palpation member 12 and a light source 14, and a second palpation assembly including a palpation member 16 and light detecting means, in the form of a miniature camera 18 for detecting light emitted by the light source 14. At least one of the first and second palpations members 12, 16 are moveable with respect to the other member, to palpate a body part 20 disposed between the palpation members.

In more detail, the first and second palpation members 12, 16 each comprise light transmitting glass plates. The palpation device 10 is mounted on suitable support means, such as a robotic or manually operated arm (not shown), and each of the first and second palpation members 12,16 are independently moveable in the directions X, X′; Y, Y′; and Z, Z′. Thus, each member 12, 16 is moveable in three mutually perpendicular planes.

This allows the first member 12 to be brought towards the second member 16, in the directions Y, Y′, to squeeze the body part 20 between the respective plate surfaces 22, 24. Simultaneously, or consecutively, the members 12,16 may be moved in the directions X, X′ to rub the body part 20 between the first and second members 12, 16. This tends to rotate or rub the body part 20 between the respective plate surfaces 22, 24. It will be understood that a similar rubbing of the body part 20 may be achieved by movement in the direction of the plane Z, Z′. These movements palpate the body part 20.

The body part 20, in the embodiment shown, comprises a length of a patient's colon, and palpating the colon 20 by squeezing/rubbing in this fashion helps in locating abnormalities such as a tumour shown schematically at 26. This is because, during palpation, the tumour 26 is squeezed and flattened as shown in FIG. 1.

The light source 14 typically comprises a light emitting diode, optical fibres or any other suitable light source, and emits light at a frequency in the visible spectrum. This light is refracted, due to the optical shape of the first member 12, and passes through the plate surface 22, through the colon 20 and into the second member 16. Thus an image is simultaneously projected on the second member 16, which is viewed by the camera 18, and the tumour 26 is highlighted as a dark, relatively dense region. Accordingly, the location and dimensions of the tumour 26 may be determined by conducting various squeezing/rubbing procedures, as described above, and by viewing the response of the colon 20 during this procedure.

The palpation device 10 may further comprise means for measuring one or more physical parameters or properties of the body part 20, such as a number of force measuring sensors (not shown). These sensors may be used for determining the force required to achieve the deformation of the body part 20 shown in FIG. 1, which may assist a surgeon in determining the location and nature of any abnormalities such as the tumour 26. In particular, the relatively dense tumour 26 would have a greater resistance to deformation by an applied force than would, for example, the relatively thin colon wall 28.

Turning now to FIG. 2, there is shown a schematic illustration of a palpation device in accordance with an alternative embodiment of the present invention, indicated generally by reference numeral 100, and shown in use in a similar fashion to the device 10 of FIG. 1. For brevity, only the features of the device 100 which differ from the device 10 of FIG. 1 will be described herein. Indeed, like components of the device 100 with the device 10 share the same reference numerals incremented by 100.

The device 100 includes a second palpation member 16 in the form of a light sensitive charge coupled device (CCD), which receives light emitted from the light source 114 projected through the colon 120, and transduces the received light into a two-dimensional pixel array. This data is outputted through an output 30 to a display such as a monitor. This allows the surgeon to monitor the response of the colon 120 during palpation.

Turning now to FIG. 3, there is shown a schematic illustration of a palpation device in accordance with a further alternative embodiment of the present invention, indicated generally by reference numeral 200. Like components of the device 200 with the device 10 of FIG. 1 share the same reference numerals, incremented by 200.

Only part of the device 200 is shown, and the device is shown out of use. A first palpation member 212 comprises a number of glass plates or sub-plates 32, 34 and 36, which are coupled together by light transmitting hinges 38 and 40. In a similar fashion, a second palpation member 216 comprises glass plates 42, 44 and 46 coupled by hinges 48 and 50.

The first member 212 is shown in a use position, where the plates 32, 34 and 36 are fully deployed, whilst the second member 216 is shown in an insertion position, with the plates 42, 44 and 46 folded about the hinges 48, 50, such that the second member 216 is of a reduced width “a” when compared to the deployed width “b” of the first member 212. This is advantageous for use in MAS surgery, where an entrance wound into a patient's body is relatively small. Each of the members 212, 216 may be deployed to the use position with a body part disposed in the space 52 defined between the member 212, 216 in a similar fashion to that illustrated in FIGS. 1 and 2.

Images obtained on each of the plates 32, 34, 36 of the first member 212, and the plates 42, 44 and 46 of the second member 216 are patched together to provide a single coherent image of the body part. This is achieved by spatial interpolation of features of the image. Typically, a line in one part of the overall image and a line in another part may be identified as being parts of the same line with a gap filled in by software. For example, a gap may exist in the overall image at the location of the hinges 38 and 40 of the first member 212. Thus small gaps in the image extending across the plates 32, 34 and 36 can be filled in by interpolation using appropriate computer software.

In a similar fashion to that described above, the members 212, 216 may be mounted on a robotic or manually operated arm for controlling the deployment and palpation movements of the device 200.

Turning now to FIGS. 4A and 4B, there are shown schematic illustrations of part of a palpation device in accordance with a still further embodiment of the present invention, indicated generally by reference numeral 300. The device 300 is similar to the device 10 of FIG. 1, and like components share the same reference numerals incremented by 300.

The device 300 is shown during palpation of a body part 320 which, as in the case of FIG. 1, may comprise a length of a patient's colon. The body part 320 is palpated in a similar fashion to that described above, to allow detection of a tumour 326, shown as a dark patch. The device 300 includes palpation members 312 and 316, and FIGS. 4A and 4B illustrate the view of tumour 326 which would be seen by the surgeon through the camera (not shown). The device 300 includes detecting means for detecting motion of at least part of the body part relative to one or both of the palpation members. In the Figures, this takes the form of a visible grid 54 provided on the surface 322 of the plate 312.

FIG. 4A shows the plates 312 and 316 in a position corresponding to that of FIG. 1, where the body part 320 has been squeezed in the direction Y-Y′ and the tumour 326 is visible. The body part 320 is then further palpated by movement of the plates 312 and 316 in opposite directions X′ and X, as shown in FIG. 4B. During this palpation movement, it can be seen that there has been no movement of the tumour 326 relative to the plate 312, as the tumour 326 is in the same position relative to the grid 54. It can therefore be determined that the tumour 326 is attached to the side of the colon adjacent the plate 312, as the tumour 326 has remained stationary with respect to the grid 54.

Turning to FIGS. 5A and 5B on the other hand, the device 300 is shown during palpation of a body part 320′ with a tumour 326′. In this case, palpation of the body part 320 in the fashion described above has caused the tumour 326′ to be displaced relative to the grid 54 on plate 312, therefore it can be determined that the tumour 326′ is attached to the inner wall of the colon adjacent to the plate 316, rather than the plate 312.

It will be understood that the provision of such a detecting means in the form of the grid 54 may equally be applied to the plates forming the palpation members of the devices 100 or 200 described above with reference to FIGS. 2 and 3. Indeed, one or both of the palpation members of each embodiment may carry such a grid.

Various modifications may be made to the foregoing within the scope of the present invention. For example, each one of the devices 10, 100 and 200 of FIGS. 1-3 may share features described and/or shown with reference to any one of the other devices.

The first and second palpation members of each device 10, 100, 200 may be of any suitable transparent or translucent material, such as a plastics material. The first and second palpation members of any of the devices 10, 100, 200 may be moveable together, or one member may be fixed whilst the other is moveable. Also, one or both of the palpation members may be moveable in only one, or two planes to palpate a body part.

In each of the embodiments described above, the light transmitted through the body part may be structured light. Light from a point source of constant intensity is unstructured. If it is used to illuminate a scene, the scene will be illuminated uniformly. In particular, if it is used to illuminate a flat uniform surface, the surface will appear uniform and featureless. Structured light, on the other hand, is not constant in intensity. The intensity varies either spatially, temporally, or both. Thus, a flat uniform surface illuminated by structured light will show features such as patterns or time varying intensities or colours. The simplest way to give light structure is by passing unstructured light through a mask. The mask imposes a spatial structure on the beam. If the beam is incident on a flat uniform surface, the surface will show the pattern made by the mask. Temporally structured light can, for example, be produced by switching an unstructured beam on and off.

The advantages of structured light include the following. Temporally structured light can be used, for example, in a stroboscope, to examine temporally changing features in an observed object. Spatially structured light can be used to highlight existing features in an observed object and thus to allow measurements to be made. For example, if light is passed through a striped mask so that it is structured into parallel lines of light and dark, it can be used to expose and measure the shape of non-flat objects. An example of light that is both spatially and temporally structured is a beam whose colour is changing with time and which is also passed through a mask. Such a beam may, by its spatial structure, be used to investigate the shape of a surface, and simultaneously, by its temporal structure, be used to investigate the optical properties of the surface material. Thus structured light may advantageously be used in the present invention to assist in determining properties of the body part under palpation.

1. A palpation device comprising:
a first palpation assembly including a first palpation member and a light source; and
a second palpation assembly including a second palpation member and a light detecting means for detecting light emitted by the light source and generating an image of a body part disposed between the first and second palpation members;
wherein at least one of the first and second palpation members is movable with respect to the other member, to palpate a body part disposed therebetween.

2. A palpation device as claimed in claim 1, further comprising means for measuring at least one physical parameter of the body part.

3. A palpation device as claimed in claim 2, wherein the physical parameter of the body part is the resistance to deformation in response to an applied force.

4. A palpation device as claimed in claim 2, wherein the measuring means comprises force measuring sensors for measuring a force applied to the body part to produce a deformation.

5. A palpation device as claimed in claim 2, wherein the measuring means comprises software for performing feature recognition and classification.

6. A palpation device as claimed in claim 1, wherein the first and second palpation members are independently moveable.

7. A palpation device as claimed in claim 1, wherein at least one of the first and second palpation members is moveable in at least two mutually perpendicular planes of motion, with respect to the body part.

8. A palpation device as claimed in claim 1, wherein both palpation members are movable in three mutually perpendicular planes of motion, with respect to the body part.

9. A palpation device as claimed in claim 1, wherein at least one of the first and second palpation members is generally planar.

10. A palpation device as claimed in claim 1, wherein the palpation device is for use in minimal access surgery (MAS).

11. A palpation device as claimed in claim 1, wherein the first palpation member further comprises a light transmitting member.

12. A palpation device as claimed in claim 11, wherein the light transmitting member is transparent.

13. A palpation device as claimed in claim 1, wherein the light source is embedded in the first palpation member.

14. A palpation device as claimed in claim 1, wherein the light source is optically coupled to the first palpation member.

15. A palpation device as claimed in claim 1, wherein the first palpation member is optically shaped to transmit a substantial part of the light emitted by the light source through a surface of said first member, the surface adapted to be located adjacent the body part.

16. A palpation device as claimed in claim 1, wherein the second palpation member further comprises a light transmitting member.

17. A palpation device as claimed in claim 16, wherein said light transmitting member is transparent.

18. A palpation device as claimed in claim 1, wherein the second palpation member further comprises a light sensitive charge coupled device (CCD).

19. A palpation device as claimed in claim 18, wherein the CCD forms the whole of the second palpation member.

20. A palpation device as claimed in claim 18, wherein the CCD is embedded in the second palpation member.

21. A palpation device as claimed in claim 18, wherein the CCD is optically coupled to the second palpation member.

22. A palpation device as claimed in claim 20, wherein the CCD is adapted to transduce received light into a 2-dimensional pixel array for output to a display device to generate the image of the body part.

23. A palpation device as claimed in claim 1, wherein the light source comprises a light emitting diode.

24. A palpation device as claimed in claim 1, wherein the light source comprises optical fibres.

25. A palpation device as claimed in claim 1, wherein the light detecting means further comprises a charge coupled device (CCD).

26. A palpation device as claimed in claim 1, wherein the light detecting means further comprises a camera.

27. A palpation device as claimed in claim 1, wherein the light detecting means further comprises an endoscope.

28. A palpation device as claimed in claim 1, wherein the light detecting means is embedded in the second palpation member.

29. A palpation device as claimed in claim 1, wherein the light detecting means is optically coupled to the second palpation member.

30. A palpation device as claimed in claim 1, wherein the first and second palpation members are moveable between an insertion position and a use position.

31. A palpation device as claimed in claim 30, wherein, in the insertion position of the first and second palpation members, the palpation device is of reduced dimensions compared to the use position of said palpation members.

32. A palpation device as claimed in claim 30, wherein the first and second palpation members each comprise at least two planar sub-plates, pivotally coupled together for movement between the insertion and use positions.

33. A palpation device as claimed in claim 32, wherein a number of separate images of the body part are obtained and are adapted to be patched together using suitable software.

34. A palpation device as claimed in claim 1, wherein the device is adapted to be mounted on a support arm having means for detecting the location and orientation of the palpation device.

35. A palpation device as claimed in claim 1, wherein the light source is adapted to emit light of a frequency in the visible spectrum.

36. A palpation device as claimed in claim 1, wherein the light source is operative to emit structured light.

37. A palpation device as claimed in claim 1, wherein the light source is operative to emit light of a frequency in the infra-red spectrum.

38. A palpation device as claimed in claim 1, wherein the device further comprises detecting means for detecting motion of at least a portion of the body part relative to at least one of the first and second palpation assemblies.

39. A palpation device as claimed in claim 38, wherein the detecting means is for detecting motion of said portion of the body part relative to the palpation member of at least one of the first and second palpation assemblies.

40. A palpation device as claimed in claim 38, wherein the detecting means comprises at least one point of reference.

41. A palpation device as claimed in claim 40, wherein the detecting means further comprises a visible grid provided on each palpation member.

42. A palpation simulation device comprising:
a first palpation assembly including a first palpation member and a light source; and
a second palpation assembly including a second palpation member and a light detecting means for detecting light emitted by the light source and generating an image of a body part disposed between the first and second palpation members;
wherein at least one of the first and second palpation members is moveable with respect to the other member, to palpate a body part disposed therebetween.

43. A method of palpating a body part, the method comprising the steps of:
providing a first palpation assembly including a first palpation member and a light source;
providing a second palpation assembly including a second palpation member and light detecting means for detecting light emitted by the light source and generating an image of a body part disposed between the first and second palpation members;
locating the first and second palpation assemblies with a body part disposed therebetween;
generating an image of said body part; and
moving at least one of the first and second palpation members relative to said other member, to palpate the body part.

44. A method of simulating palpation of a body part, the method comprising the steps of:
providing a first palpation assembly including a first palpation member and a light source;
providing a second palpation assembly including a palpation member and light detecting means for detecting light emitted by the light source and generating an image of a body part disposed between the first and second palpation members;
locating the first and second palpation assemblies with a body part disposed therebetween;
generating an image of said body part; and
moving at least one of the first and second palpation members relative to said other member, to palpate the body part.
